(19) United States
(12) Patent Application Publication
(10) Pub. No.: US 2007/0232690 A1
(43) Pub. Date: Oct. 4, 2007

(54) Composition for inducing differentiation of leukemic cells and application thereof

(75) Inventors: Yu-Jen Chen, Taipei (TW); Hui-Fen Liao, Taipei (TW); Yueh-Hsiung Kuo, Taipei (TW)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: Mackay Memorial Hospital, Taipei (TW)

(21) Appl. No.: 11/391,581

(22) Filed: Mar. 28, 2006

(51) Int. Cl. A61K 31/343

(52) U.S. Cl. 514/469; 549/468

(57) ABSTRACT

Disclosed is a medical composition for inducing differentiation of leukemic cells, which can induce leukemic cells to differentiate into monocytes and macrophages, and block the abnormal proliferation of leukemic cells through a PKC-independent pathway. In addition, the medical composition cans also downregulate c-myc gene expression in the leukemic cells, and upnregulate c-fms, c-fos and c-jun gene. The medical composition comprises a compound represented by the general formula (I) shown below: 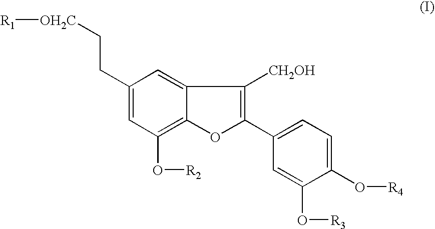
wherein R₁, R₂, and R₃ represent —H, a linear or branched alkyl group with 1 to 3 carbon atoms, and R₄ represents —H or a benzyl group.

### BACKGROUND OF THE INVENTION

1. Field of the Invention

The present invention relates to a medical composition for leukemia treatment, especially relates to a leukemic cell differentiation inducing composition for leukemia treatment.

2. The Prior Arts

Leukemia is a hematological malignancy characterized by a differentiation block that leads to an accumulation of immature cells. At the same time, losing normal mature cells can cause malfunction. Leukemia can be divided into 4 main types: acute lymphoblastic leukemia (ALL), acute myeloid leukemia (AML), chronic lymphoblastic leukemia (CLL), and chronic myeloid leukemia (CML). Among them, AML is a chiefly adult disease, and the bone marrow cells being affected include granulocytes, monocytes, red blood cells and platelets, with granulocytes being the most affected.

Chemotherapy is known to be the main treatment for leukemia. Chemotherapy uses strong drugs to kill immature white blood cells. Patients receiving chemotherapy may have unnecessary side effects, such as low white blood cells caused by inhibiting bone marrow, anemia, infection, bleeding, or other serious problems and so on.

As mentioned above, chemotherapy causes many side effects that make patients uncomfortable and threatening to life. To solve the problem, many studies have devoted to develop differentiation induction treatment of leukemia to induce immature cells to be differentiated into functionally mature white blood cells. Several differentiation inducing agents have been known to induce immature cells to differentiate into mature cells. For example, all-transretinoic acid (ATRA) and arsenic trioxide (As₂O₃) have been reported to cause differentiation of immature cells into morphologically and functionally mature granulocytes.

Monocytes and macrophages play a major role in innate immune response of human immune system. Both of them and their derived dendritic cells are main groups of antigen-presenting cells. Previous known differentiation inducing agents cannot induce immature cells to differentiate into monocytes and macrophages effectively. Therefore, it will be of great clinical importance and value if a medical composition, which can induce differentiation of leukemic cells into monocytic and macrophages and block the abnormal proliferation of leukemic cells is developed.

### SUMMARY OF THE INVENTION

A primary object of the present invention is to provide a compound for inducing differentiation of leukemic cells in order to induce the differentiation of immature white blood cells into the functionally mature cells.

A compound for inducing differentiation of leukemic cells according to the invention is represented by the following general formula (I) shown below: 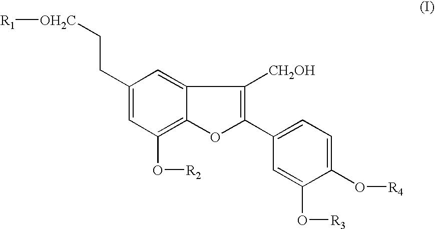
wherein R₁, R₂, and R₃ represent —H, a linear or branched alkyl group with 1 to 3 carbon atoms, and R₄ represents —H or a benzyl group.

The compound in the invention exhibits the induction of leukemic cell differentiation into monocytes and macrophages, and growth inhibitory effects of abnormal proliferation on leukemic cells, which is not related to activation of protein kinase C. In addition, the amplification of c-myc gene in leukemic cells is downregulated, and the genes such as c-fms, c-fos and c-jun are upregulated by the compound of the invention.

Another object of the present invention is to provide a medical composition for inducing differentiation of leukemic cells, which comprises a compound represented by the following general formula (I) shown below and a pharmacologically acceptable carrier: 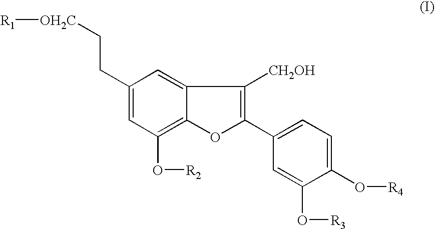
wherein R₁, R₂, and R₃ represent —H, a linear or branched alkyl group with 1 to 3 carbon atoms, and R₄ represents —H or a benzyl group.

A further object of the present invention is to provide a method for treating leukemia in a subject in need thereof, which comprises administering to the subject a therapeutically effective amount of a medical composition comprising a compound for inducing differentiation of leukemic cells and a pharmacologically acceptable carrier, wherein the compound is represented by the following general formula (I) shown below: 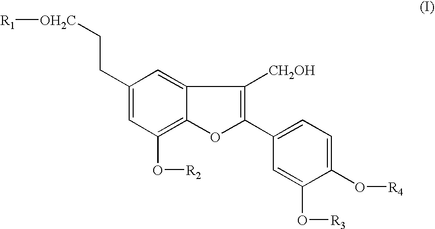
wherein R₁, R₂, and R₃ represent —H, a linear or branched alkyl group with 1 to 3 carbon atoms, and R₄ represents —H or a benzyl group.

The present invention is further explained in the following embodiment illustration and examples. The present invention disclosed above is not limited by these examples. The present invention may be altered or modified by people skilled in the art and all such variations are within the scope and spirit of the present invention.

### BRIEF DESCRIPTION OF THE DRAWINGS

FIG. 1 shows dose-dependent growth inhibition of human leukemic HL-60 cells treated with various concentrations of 278E.

FIG. 2 illustrates images under a microscope of HL-60 cells after 278E induction of differentiation; (A) untreated cells, (B) 278E-treated cells.

FIG. 3 shows the percentages of differentiated cells induced by 278E at various concentrations.

FIG. 4 shows the gene expression in the 278E-treated HL-60 cells. (A) c-myc gene; (B) c-fms gene; (C) c-jun gene; (D) c-fos gene.

### DETAILED DESCRIPTION OF THE PREFERRED EMBODIMENT

A compound for inducing differentiation of leukemic cells according to the invention is represented by the following formula (I): 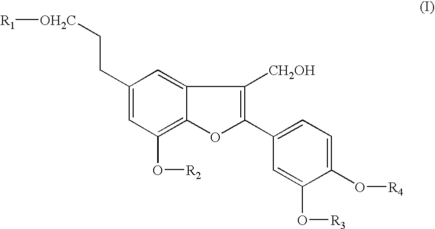
wherein R₁, R₂, and R₃ represent —H, a linear or branched alkyl group with 1 to 3 carbon atoms, and R₄ represents —H or a benzyl group.

The compound of the invention can be prepared from known synthetic organic chemical technology, or partial chemical modification on functional groups of the compound as represented by formula (II) isolated from Salvia miltiorrhiza Bunge.

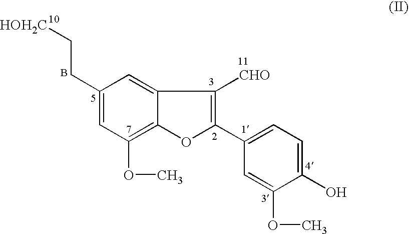

Besides inhibiting proliferation of leukemic cells, the compound of the invention can also induce differentiation in leukemic cells to the mature monocytes and macrophages.

Treatment with the compound of the invention resulted in induction of little superoxide-producing immature cells into more superoxide-producing mature cells. In addition, the compound of the invention can also induce immature leukemic cells differentiated into mature antigen bearing cells, that is, functionally mature cells. The aforementioned antigens include, but are not limited to: CD11b, CD14 and CD68.

Activation of protein kinase C (PKC) and c-myc protein expression are associated with the induction of monocytic differentiation in tumor necrosis factor-a-treated HL-60 cells. According to previous report, RACK1 (receptor for activated C kinase-1) is involved in PKC-mediated signal transduction and subcellular PKC localization. Amplification of c-myc in HL-60 cells is downregulated during monocytic differentiation. Expression of protooncogenes such as c-fms, c-fos and c-jun is also involved in monocytic differentiation of HL-60 cells. The compound of the invention can cause a downregulation of c-myc as well as an upregulation of cfms, c-fos and c-jun in leukemic cells.

On the other hand, the pretreatment of PKC inhibitor such as staurosporine and H7 to HL-60 cells do not suppress the proliferation-inhibiting and differentiation-inducing activities of the compound of the invention. Namely, the compound of the invention is not a protein kinase C activator. The induction of leukemic cells to differentiate into monocytes and macrophages is not related to the pathway of protein kinase C.

After the aforementioned explanation is read, people skilled in the art will understand that the compound of the invention can be added with a pharmacologically acceptable carrier to form a medical composition. Also, the medical composition can be administered to a subject suffered from leukemia a therapeutically effective amount to induce differentiation of leukemic cells. Any suitable route of administration may be employed for providing the subject with a therapeutically effective amount of the medical composition. For example, oral, rectal, parenteral (subcutaneous, intramuscular, intravenous), transdermal, and like forms of administration may be employed. Dosage forms include tablets, troches, dispersions, suspensions, solutions, capsules, patches, and the like.

Leukemia indicated in the invention includes: acute lymphocytic leukemia (ALL), acute myeloid leukemia (AML), chronic lymphoblastic leukemia (CLL), and chronic myeloid leukemia (CML), wherein AML is especially indicated.

The compounds indicated in the invention are represented by formula (IIII) (namely 278E hereafter) to be one of the examples of formula (I) to describe the characteristics of the compounds in the invention, and need not be limited to the example.

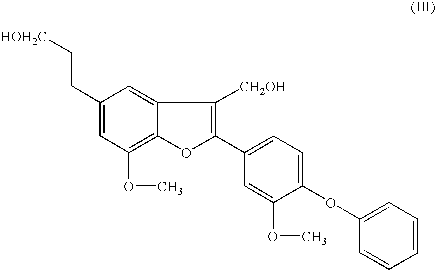

### EXAMPLE 1

### Assay for Cell Growth Inhibition

The human myeloid leukemic cell line HL-60 is known to be induced by several biological agents or chemical drugs to differentiate into mature cells. The results of differentiation can be cell morphological changes, cell chemical changes or functionally mature changes. Therefore, HL-60 cells were often applied in many differentiation studies of leukemic cells. The present invention used the human myeloid leukemic cell line HL-60 (ATCC, Manassas, Va.) to study the cell differentiation inducting ability of 278E.

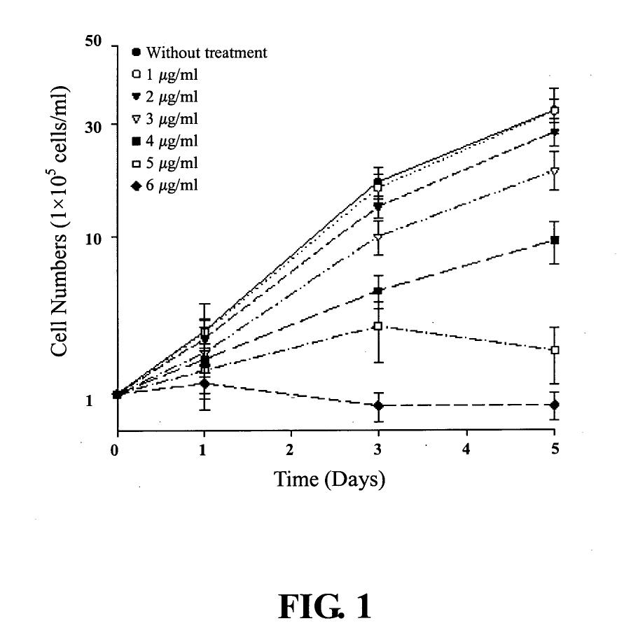

The HL-60 cells were cultured in RPMI 1640 medium containing 10% fetal bovine serum and maintained in an exponential growth state. For assay of cytotoxicity, cells were incubated at an initial concentration of 1×10⁵ cells/ml in the presence of various concentrations of 278E compounds (1-6 μg/ml) for 1-5 days. The concentration yielding 50% inhibition (IC₅₀) was calculated to be 3.48 μg/ml. The numbers of viable cells were counted using the Trypan blue dye exclusion test and were shown in FIG. 1.

The growth of HL-60 cells was inhibited significantly by 278E in a dose- and time-dependent manner (FIG. 1). A concentration of 1 μg/ml 278E had a minimal effect on cell viability compared with untreated cells. However, concentrations up to 5 μg/ml caused marked growth inhibition (85.5±2.8% on day 3 and 91.7±1.7% on day 5).

### EXAMPLE 2

### Maturation Profiles

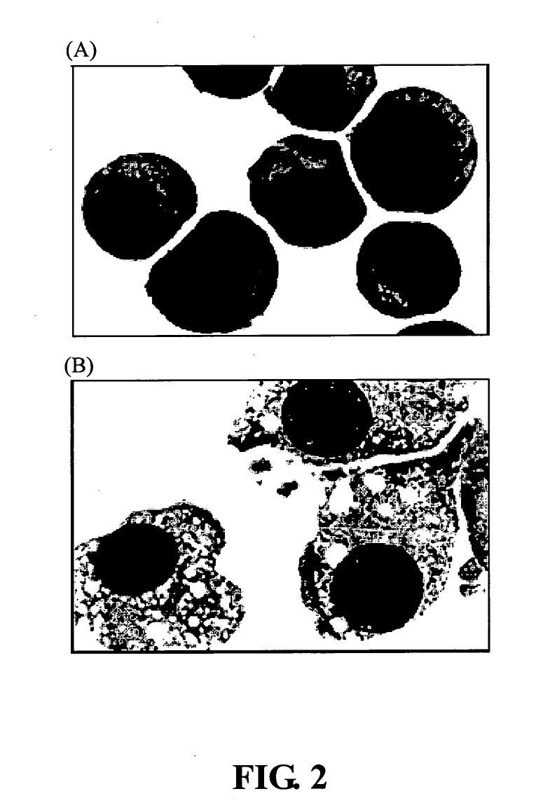

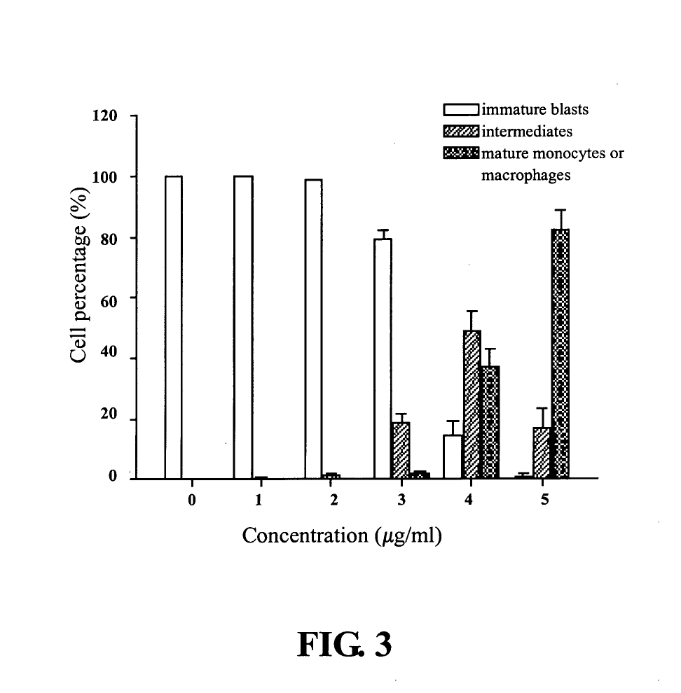

After 5 days of treatments (1-5 μg/ml of 278E) as described in Example 1, HL-60 cells were collected and cytocentrifuged onto a microscope slide using a Cytospin, stained with Wright's stain and observed under an inverted microscope (Olympus) with a magnification of 1000×. Based on morphology, the cells were classified as: (1) immature blasts, (2) intermediates, or (3) mature monocytes or macrophages, and the ratio was counted respectively. The results were shown in FIG. 2 and FIG. 3.

Untreated HL-60 cells were morphologically myeloblast-like cells, with a round cell contour, cytoplasm containing granules, and an ovoid-shaped nucleus with some nucleoli (FIG. 2A). After 5 days of treatment with 278E, many cells exhibited changes in characteristics, such as a lower nucleus/cytoplasm ratio, a less basophilic cytoplasm, scanty nucleoli, evident pseudopodia (FIG. 2B).

In addition, the percentage of mature monocyte/macrophage was 0% in untreated HL-60 cells and 82.0±6.3% after 5 days of 278E treatment (FIG. 3).

### EXAMPLE 3

### Assay for Superoxide Production

It has been known that immature blast cells had little superoxide production. The differentiation of cells is further evidenced in changes of superoxide production. Cells collected from example 1 after treatment with 278E for 5 days were collected and resuspended in RPMI 1640 medium at a concentration of 1×10⁶ cells/ml and incubated for 30 min at 37° C. with an equal volume of nitroblue tetrazolium test stock solution [containing 2 mg of NBT and 1 μM of phorbol myristate acetate per ml of phosphate buffered saline]. Cells were collected and cytocentrifuged onto a microscope slide using cytospin. Cytospin preparations were counter-stained with 0.5% safranin O. The percentage of formazan-containing cells (based on counting 200 cells) was assessed microscopically. The result is shown in Table 1.

Table 1 showed that treatment with 5 μg/ml 278E for 5 days resulted in a marked increase in the percentage of superoxide-producing cells.

### EXAMPLE 4

### Assay for Differentiation Antigens

An indirect immunofluorescence method was employed in the invention to detect the expression of monocyte-associated antigens on the surface of HL-60 cells after induction of differentiation. 278E-treated cells collected from day 5 cultures were treated with primary monoclonal antibodies, washed with PBS and then exposed to a solution containing fluorescein isocyanate (FITC) conjugated secondary antibody, goat F(ab′)₂ antimouse IgG (Cappel, Cochranville, Pa.). The aforementioned monoclonal antibodies against human monocytes included anti-CD 11b, anti-CD14 (Serotec, Oxford, UK), and anti-CD68 (Dakopatts, Glostrup, Denmark). The percentage of positive cells analyzed with a FACScan flow cytometer (Becton Dickenson, Franklin Lakes, N.J.) and Lysis II software (Becton Dickinson) is shown in Table 2.

Table 2 shows that the percentage of CD11b-, CD14- and CD68-bearing cells was low (<3%) in untreated HL-60 cells. As the dose of 278E increased to 5 μg/ml, the percentage of CD14-positive cells increased to 63.6±8.9%. Similar results were observed in HL-60 cells expressing CD11b and CD68.

### EXAMPLE 5

### Induction of Differentiation by Various Combination Treatments

The differentiation of HL-60 into monocytes/macrophages after various treatments were detected and assessed as described in Example 2. These combination treatments included: (1) no treatment, (2) PKC inhibitor Staurosporine, (3) PKC inhibitor H7, (4) protein kinase activator TPA, (5) PKC inhibitor Staurosporine and TPA, (6) H7 and TPA, (7) 278E, (8) Staurosporine and 278E, and (9) H7 and 278E. The results are shown in Table 3.

Table 3 demonstrates that the pretreatment of PKC inhibitor H7 and staurosporine to HL-60 cells suppressed the differentiation-inducing activity of TPA, but not of 278E. PKC inhibitors per se had no differentiation-inducing effect on HL-60 cells.

### EXAMPLE 6

### Expression of Proto-Oncogenes

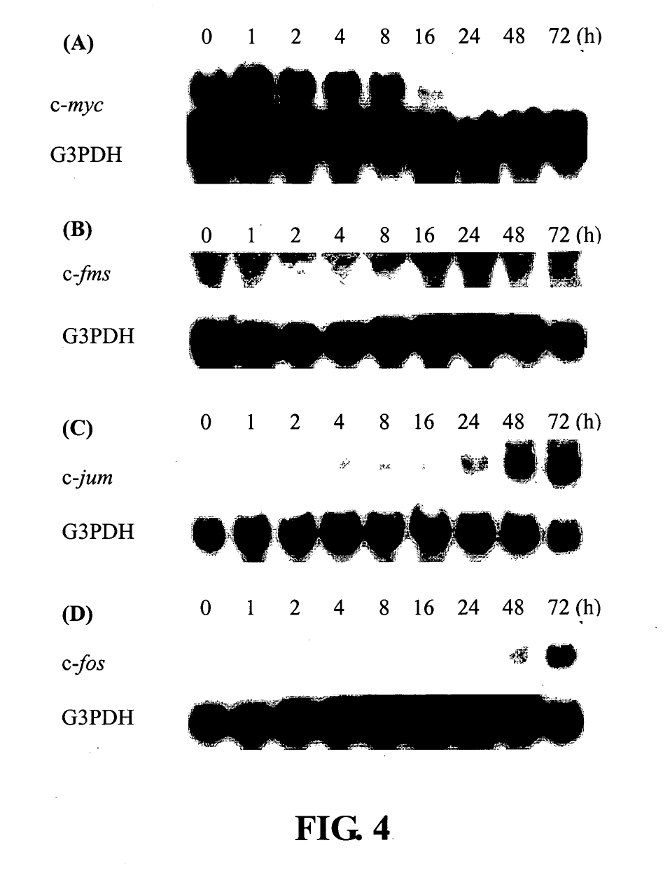

Total RNA was isolated from HL-60 cells using an RNAzol B kit (Biotecx, Galveston, Tex.) and quantified spectrophotometrically. Total cellular RNA (20 μg) from control or 278E-treated cells was electrophoresed on a 1.2% agarose gel and transferred to a nylon membrane. The blots were probed with radiolabeled cDNA of c-myc, c-fms, c-fos and c-jun. Glyceraldehyde-3-phosphate dehydrogenase (G3PDH) was used as an internal control. The results are shown in FIG. 4.

As shown in FIG. 4, expression of c-myc was markedly suppressed in HL-60 cells after 16 hour treatment with 6 μg/ml 278E (FIG. 4A). On the contrary, expression of c-fms, c-jun and c-fos was enhanced respectively after treatment.

TABLE 1
NBT reduction of HL-60 cells after 278E treatment

| Concentration of 278E (μg/ml) | Positive cells (%) |
|---|---|
| 0 | 0 |
| 1 | 0.3 ± 0.3 |
| 2 | 1.8 ± 1.2 |
| 3 | 9.3 ± 2.8 |
| 4 | 59.0 ± 12.6 ^{a} |
| 5 | 95.5 ± 1.8 ^{a} |

^{a} p < 0.05

TABLE 2
Surface marker expression on HL-60 cells induced by 278E

| Concentration | Positive cells (%) | | |
|---|---|---|---|
| of 278E (μg/ml) | CD11b | CD14 | CD68 |
| 0 | 1.9 ± 0.4 | 2.4 ± 0.7 | 1.9 ± 0.4 |
| 1 | 1.8 ± 0.5 | 1.5 ± 0.4 | 1.4 ± 0.4 |
| 2 | 2.6 ± 0.6 | 2.1 ± 0.6 | 1.8 ± 0.4 |
| 3 | 18.1 ± 6.5 ^{a} | 5.8 ± 2.8 | 5.6 ± 2.1 |
| 4 | 62.1 ± 10.4 ^{a} | 47.7 ± 10.6 ^{a} | 34.5 ± 9.9 ^{a} |
| 5 | 79.6 ± 9.8 ^{a} | 63.6 ± 8.9 ^{a} | 53.7 ± 10.0 ^{a} |

^{a} p < 0.05

TABLE 3
Induction of monocytic differentiation
by various combination treatments

| | monocytes/macrophages |
|---|---|
| Treatment | (%) |
| No | 0 |
| Staurosporine 10 nM | 0 |
| H7 15 μM | 0 |
| TPA 10 ng/ml | 76.8 ± 7.2 |
| Staurosporine 10 nM and TPA 10 ng/ml | 35.0 ± 2.3 |
| H7 15 μM and TPA 10 ng/ml | 30.2 ± 4.5 |
| 278E 6 μg/ml | 56.1 ± 6.2 |
| Staurosporine 10 nM and 278E 6 μg/ml | 55.6 ± 4.7 |
| H7 15 μM and 278E 6 μg/m | 51.3 ± 5.3 |

Note:
Staurosporine and H7 are PKC inhibitors; TPA is a protein kinase activator.

What is claimed is:

1. A medical composition for inducing differentiation of leukemic cells, comprising a compound represented by the general formula (I) and a pharmacologically acceptable carrier: 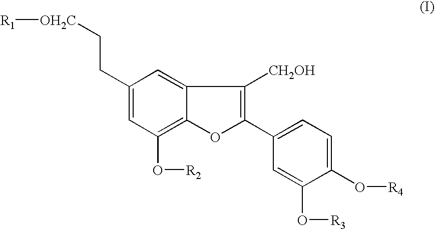
wherein R₁, R₂, and R₃ represent —H, a linear or branched alkyl group with 1 to 3 carbon atoms, and R₄ represents —H or a benzyl group.

2. The medical composition as claimed in claim 1, wherein the compound is represented by formula (III).

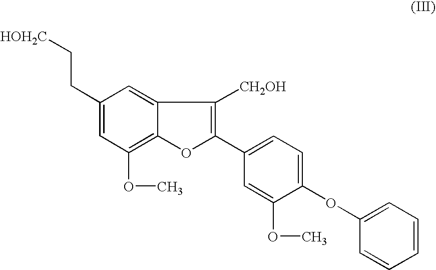

3. The medical composition as claimed in claim 1, wherein the leukemia is acute lymphocytic leukemia.

4. The medical composition as claimed in claim 1, wherein the medical composition can induce leukemic cells to differentiate into mature cells.

5. The medical composition as claimed in claim 4, wherein the mature cells are selected from the group consisting of monocytes and macrophages.

6. The medical composition as claimed in claim 4, wherein the mature cells can produce superoxide.

7. The medical composition as claimed in claim 4, wherein the mature cells are antigen-bearing cells.

8. The medical composition as claimed in claim 7, wherein antigens of the antigen-bearing cells are selected from the group consisting of CD11b antigen, CD14 antigen and CD68.

9. The medical composition as claimed in claim 1, wherein the medical composition can downregulate c-myc gene in the leukemic cells.

10. The medical composition as claimed in claim 1, wherein the medical composition can upnregulate expression of genes selected from the group consisting of c-fms, c-fos and c-jun genes in the leukemic cells.

11. A compound for inducing differentiation of leukemic cells, is represented by the general formula (I) as claimed in claim 1: 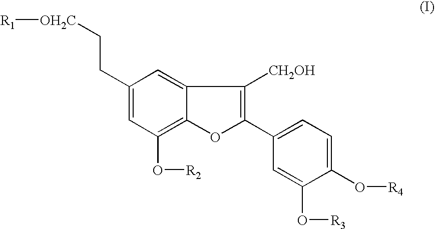
wherein R₁, R₂, and R₃ represent —H, a linear or branched alkyl group with 1 to 3 carbon atoms, and R₄ represents —H or a benzyl group.

12. The compound as claimed in claim 11, which is represented by formula (III).

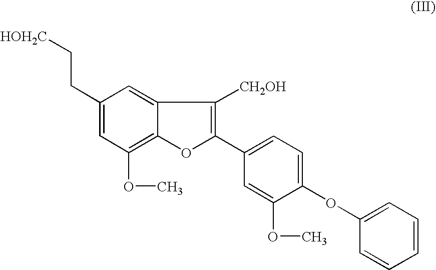

13. The compound as claimed in claim 11, wherein the leukemia is acute lymphocytic leukemia.

14. The compound as claimed in claim 11, wherein the compound can induce leukemic cells to differentiate into mature cells.

15. The compound as claimed in claim 14, wherein the mature cells are selected from the group consisting of monocytes and macrophages.

16. The compound as claimed in claim 14, wherein the mature cells can produce superoxide.

17. The compound as claimed in claim 14, wherein the mature cells are antigen-bearing cells.

18. The compound as claimed in claim 17, wherein antigens of the antigen-bearing cells are selected from a group consisting of CD11b antigen, CD14 antigen and CD68.

19. The compound as claimed in claim 11, wherein the compound can down-regulate c-myc gene in the leukemic cells.

20. The compound as claimed in claim 11, wherein the compound can upnregulate expression of genes selected from the group consisting of c-fms, c-fos and c-jun genes in the leukemic cells.

21. A method for treating leukemia in a subject in need thereof, comprising administering to the subject a therapeutically effective amount of the medical composition as claimed in claim 1.

22. A method for treating leukemia in a subject in need thereof, comprising administering to the subject a therapeutically effective amount of the compound as claimed in claim 11.
